(19) United States
(12) Patent Application Publication
(10) Pub. No.: US 2025/0387245 A1
(43) Pub. Date: Dec. 25, 2025

(54) COVERED STENT

(71) Applicant: BCM Co., LTD., Gyeonggi-do (KR)

(72) Inventor: Byung Cheol MYUNG, Gyeonggi-do (KR)

(21) Appl. No.: 19/246,716

(22) Filed: Jun. 24, 2025

(30) Foreign Application Priority Data
Jun. 25, 2024 (KR) 10-2024-0082445

(51) Int. Cl. A61F 2/90

(52) U.S. Cl. CPC A61F 2/90; A61F 2210/0014; A61F 2230/0069

(57) ABSTRACT

Disclosure relates to a covered stent preventing a bent part due to poor coating of a polytetrafluoroethylene (PTFE) film part from being exposed and sticking or causing friction to a lumen of the body at a lesion area. The covered stent includes a cylindrical stent made of shape memory alloy wires intersected together into a hollow cylindrical mesh shape to form multiple space parts, and having multiple bent parts formed on each end along circumference, a film part formed by bonding first and second PTFE tapes on inner and outer surfaces of the stent with heat and pressure, and a PTFE protection member provided at each stent end, having a hollow cylindrical shape and having a groove formed at a thickness portion to cover each bent part with an end of the cylindrical stent inserted therein, and bonded to an outer portion of the film part with heat and pressure.

### CROSS REFERENCE TO RELATED APPLICATION

The present application claims priority to Korean Patent Application No. 10-2024-0082445, filed Jun. 25, 2024, the entire contents of which are incorporated herein for all purposes by this reference.

### BACKGROUND OF THE INVENTION

### Field of the Invention

The present disclosure relates to a covered stent used to expand a passage with a stenotic or occlusive lesion area occurring in a lumen of the human body.

### Description of the Related Art

Generally, when a stenotic or occlusive lesion occurs in a lumen of the body, such as the urethra, pancreatic duct, etc., of the human body, the inherent function of the lumen to move fluids is decreased.

Therefore, a stent is inserted into a portion with a stenotic or occlusive lesion to expand the narrowed lumen.

In this respect, patent document 1 provides a method for manufacturing a stent graft and a stent graft. The method for manufacturing a stent graft includes: manufacturing a stent having a hollow cylindrical body formed by weaving crossed wires of super-elastic shape memory alloy; inserting the stent into an outer portion at which an inner artificial blood vessel layer of a polytetrafluoroethylene (PTFE) material is formed by wrapping a P artificial blood vessel diagonally around an outer surface of a suspension rod, and forming an outer artificial blood vessel layer of a PTFE material by wrapping an artificial blood vessel diagonally around n outer portion of the stent and then inserting a silicone tube to prepare bonding; mounting the prepared suspension rod into a vacuum heating device and then covering the stent from the outside and inside inner and outer by the artificial blood vessel layers located inside and outside the stent so that the inner and outer artificial blood vessel layers are vacuum-adsorbed toward the stent, bonding the inner and outer artificial blood vessel layers integrally with the stent as the inner and outer artificial blood vessel layers are thermally fused together by heating.

However, in patent document 1, as shown in FIG. 1, multiple bent parts formed along the circumference of either end of the stent may prevent the PTFE inner and outer artificial vessel layers from being properly coated.

Therefore, the sharp bent parts may be exposed at the PTFE inner and outer artificial vessel layers.

For this reason, as shown in FIG. 2, when the stent with the PTFE inner and outer artificial vessel layers is used in a narrowed or blocked lesion area in a lumen of the body, the sharp bent parts may repeatedly stick the lumen in the body or cause friction, causing irritation and inflammation.

### DOCUMENT OF RELATED ART

### Patent Document

Patent Document 1: Korean Patent Application Publication No. 10-2015-0052719 (May 14, 2015)

### SUMMARY OF THE INVENTION

Accordingly, an objective of the present disclosure is to provide a covered stent different from a conventional stent, the covered stent including a PTFE protection material for covering a sharply bent part of the covered stent at which a polytetrafluoroethylene (PTFE) film part is formed, thereby preventing exposure of the sharply bent part occurring with poor coating in the PTFE film part, preventing the sharply bent part from repeatedly sticking or causing friction against a lumen in the body with a lesion area.

In order to achieve the above-described objective, according to the present disclosure, there is provided a covered stent, the covered stent including: a cylindrical stent formed from shape memory alloy wires that are woven or intersected together into a hollow cylindrical mesh shape to form multiple space parts within the wires, and having multiple bent parts formed on each end along circumference; a film part formed by bonding first and second polytetrafluoroethylene (PTFE) tapes, the tapes being respectively formed on inner and outer surfaces of the cylindrical stent, together with heat and pressure; and a polytetrafluoroethylene (PTFE) protection member provided at each end of the cylindrical stent, having a hollow cylindrical shape and having a groove formed at a thickness portion to cover each bent part as an end of the cylindrical stent is inserted into the groove, and bonded to an outer portion of the film part with heat and pressure,

According to the present disclosure, the sharply bent part of the covered stent at which the PTFE film part is formed is covered by the PTFE protection member, and the PTFE protection member can prevent exposure of the sharply bent part occurring with poor coating of the PTFE film part.

For this reason, the PTFE protection member can prevent the sharply bent part of the covered stent from repeatedly sticking or causing friction against the lumen of the body at a lesion area, and prevent irritation and inflammation.

According to the present disclosure, the sharply bent part formed at both ends of the covered stent can be covered by the PTFE protection member formed in advance.

Furthermore, a portion of the PTFE protection member is inserted into or fitted over the cylindrical stent, and a remaining portion is folded, so that a sharp bent part formed on each end of the covered stent can be covered by the PTFE protection member.

In other words, the PTFE protection member can be installed in various ways at each end of the covered stent.

According to the present disclosure, with the second PTFE tape having a helical shape, some of hook parts and cross parts formed by the woven or intersected wires can be prevented from being held by the first and second PTFE tapes of the film part.

For this reason, the covered stent has the adaptability due to free hook parts and free cross parts, and the covered stent can be easily deformed into a curved shape in response to a curved lumen of the body.

According to the present disclosure, the second PTFE tape having a helical shape to allow unblocked space part of the covered stent to hold a lesion area and a portion of a lumen of the body inserted therein, so that the covered stent can be obstructed by the lesion area and the lumen of the body inserted into inserted into the space part. For this reason, due to shaking of the body and external force occurring from the outer part, the covered stent can be prevented from being removed from the lesion area.

### BRIEF DESCRIPTION OF THE DRAWINGS

FIGS. 1 and 2 are a front view and an operational condition view of a conventional stent,

FIGS. 3 to 13 are front, partially expanded, and detailed views of a covered stent according to an embodiment of the present disclosure,

FIGS. 14 and 15 are operational condition views of the covered stent according to the embodiment of the present disclosure,

FIGS. 16 to 20 are front, partially expanded, and detailed views of the covered stent according to a first modified example of the embodiment of the present disclosure, and

FIGS. 21 and 22 are operational condition views of the covered stent according to the first modified example of the embodiment of the present disclosure.

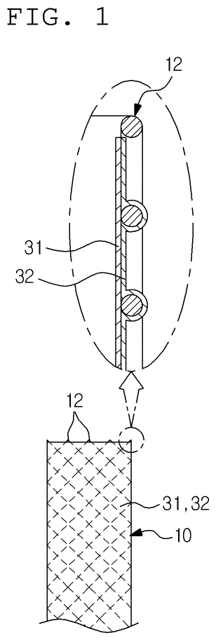

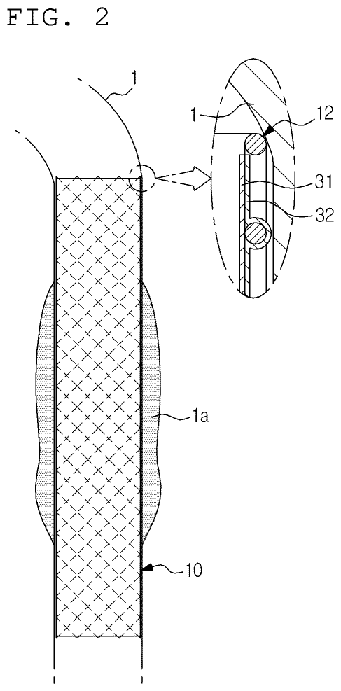

### DETAILED DESCRIPTION OF THE DISCLOSURE

Accordingly, exemplary embodiments of the present disclosure will be described in detail with reference to the accompanying drawings as follows.

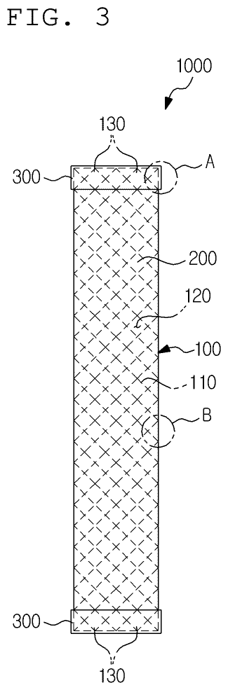

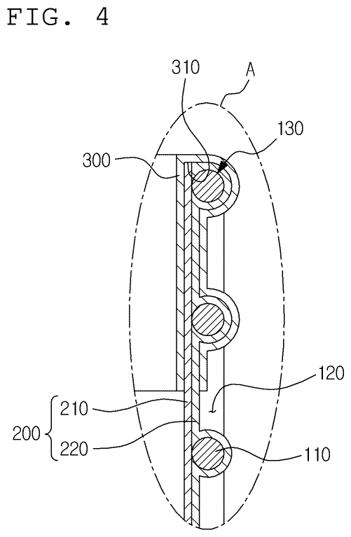

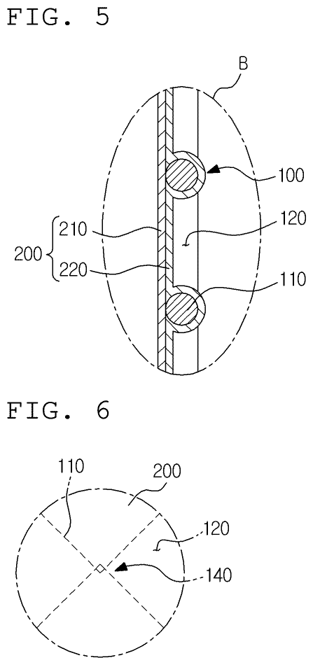

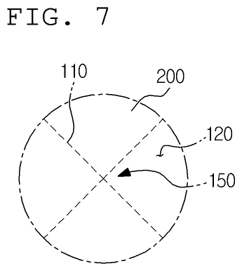

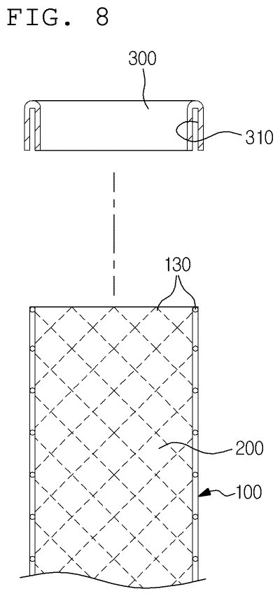

As shown in FIGS. 3 to 15, according to the embodiment of the present disclosure, a covered stent 1000 is inserted into a lumen 1 in the body, such as urethra, pancreatic duct, etc. of the human body, by a stent delivery system, and is used to expand the lesion area la of narrowing or blocking occurring in the lumen 1.

At this point, the stent delivery system includes: a first handle to which an outer tube moving along the lumen 1 in the body is connected; a second handle moving behind the first handle; an inner tube connected to the second handle and inserted into an outer tube movably, and having a mounting space at one portion for the stent of the present disclosure to be forcibly mounted to the outer tube; and a guide tip connected to the inner tube and exposed from the outer tube.

The covered stent 1000 includes a cylindrical stent 100 made of shape memory alloy wires 110 woven or intersected into a mesh hollow cylindrical shape to form multiple space parts 120 within the wires 110, having hook parts 140 and cross parts 150 around the space parts 120, and having multiple bent parts 130 along the circumference at both ends.

At this point, the hook parts 140 and the cross parts 150 are formed by weaving or intersecting the wires 110.

The covered stent 1000 includes a film part 200, the film part 200 being formed by bonding first and second polytetrafluoroethylene (PTFE) tapes 210 and 220 formed on inner and outer surfaces of the cylindrical stent 100 together with heat and pressure.

At this point, the film part 200 is provided at the cylindrical stent 100 by wrapping the first PTFE tape 210 around an outer surface of a jig, inserting the jig into the cylindrical stent 100, wrapping the second PTFE tape 210 around an outer surface of the cylindrical stent 100, and heating and compressing the cylindrical stent 100 with the jig inserted therein until the first and second PTFE tapes 210 and 220 are bonded together.

Furthermore, the first and second PTFE tapes 210 and 220 are formed throughout the entire inner and outer surfaces of the cylindrical stent 100, and the multiple space parts 120 of the cylindrical stent 100 are blocked by the film part 200.

The covered stent 1000 includes a pair of PTFE protection members 300, the pair of protection members 300 being located at both ends of the cylindrical stent 100, having a hollow cylindrical shape and having a ring-shaped groove 310 formed at a thickness portion to cover each bent part 130 when an end of the cylindrical stent 100 is inserted therein, and bonded to an outer portion of the film part 200 by heat and pressure.

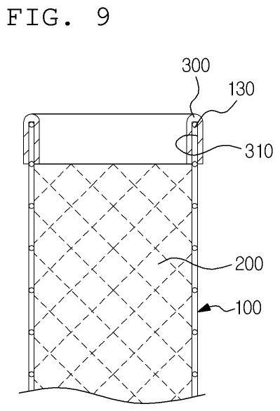

As shown in FIGS. 8 and 9, the PTFE protection members 300 are located at both ends of the cylindrical stent 100 at which the film part 200 is formed, and an end of the cylindrical stent 100 is inserted into the groove 310 and covers each bent part 130.

Furthermore, the jig is inserted into the cylindrical stent 100 with the PTFE protection members 300 coupled to both ends of the cylindrical stent 100, and then the PTFE protection members 300 and the film part 200 are heated and compressed until they are bonded together, and therefore the stent 1000 of according to the embodiment of the present disclosure is completed.

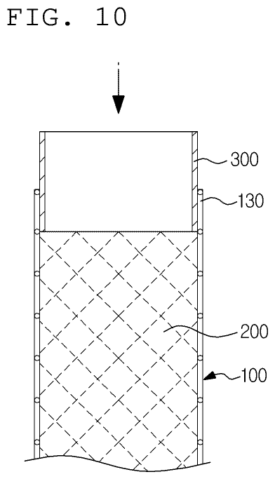

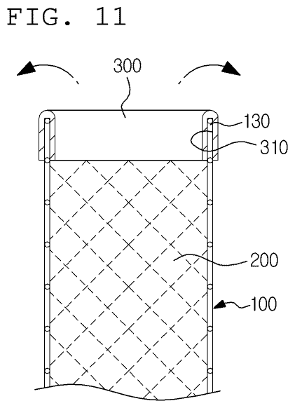

As shown in FIGS. 10 and 11, each PTFE protection member 300 has a hollow cylindrical shape, a portion of each PTFE protection member 300 is inserted into the cylindrical stent 100 at each end of the cylindrical stent 100, a remaining portion thereof is folded externally to cover each end of the cylindrical stent 100 to form the groove 310 for the cylindrical stent 100 to be inserted into the groove 310 and the bent parts 130 to be covered, and then each PTFE protection member 300 may be bonded to the film part 200 by heat and pressure with the jig inserted into the cylindrical stent 100.

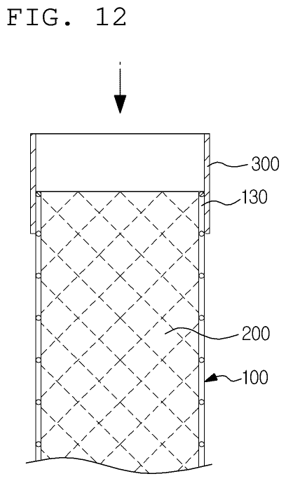

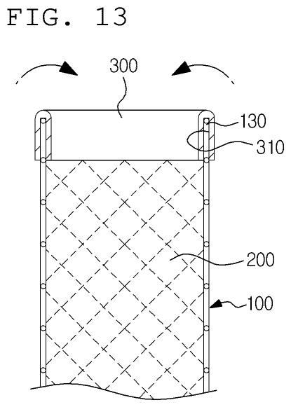

As shown in FIGS. 12 and 13, each PTFE protection member 300 has a hollow cylindrical shape, a portion of each protection member 300 is fitted over an outer portion of the cylindrical stent 100 at each end of the cylindrical stent 100, a remaining portion of each protection member 300 is folded inwardly to cover each end of the cylindrical stent 100 so that the groove 310 is formed to cover each bent part 130 as an end of the cylindrical stent 100 is inserted into the groove, and heat and pressure are applied to bond the protection member 300 to the film part 200 with the jig inserted in the cylindrical stent 100.

According to the embodiment of the present disclosure, the operation of the covered stent 1000 is described as follows.

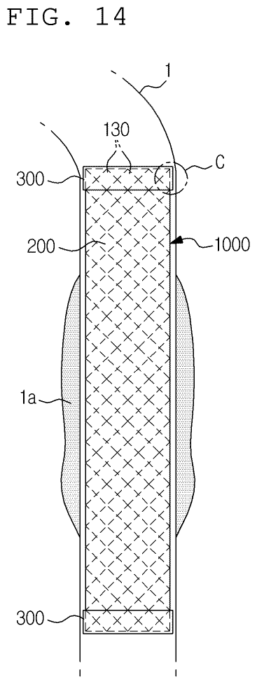

As shown in FIGS. 14 to 15, according to the embodiment of the present disclosure, the covered stent 1000 is inserted into the lumen 1 in the body, such as urethra, pancreatic duct, etc. of the human body, by the stent delivery system, and is used to expand the lesion area la of narrowing or blocking occurring in the lumen 1.

At this point, since the bent parts 130 of the covered stent 1000 are wrapped by the PTFE protection members 300 without being completely wrapped by the first and second PTFE tapes 210 and 220 of the film part 200, the bent parts 130 may not stick or cause friction against the lumen 1 in the body.

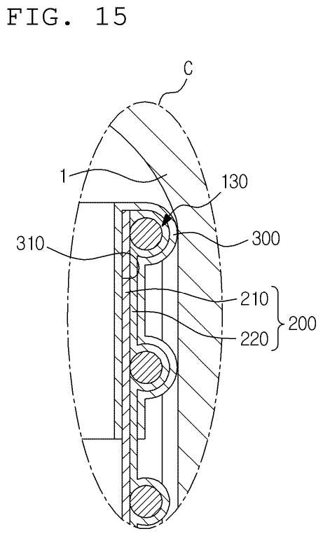

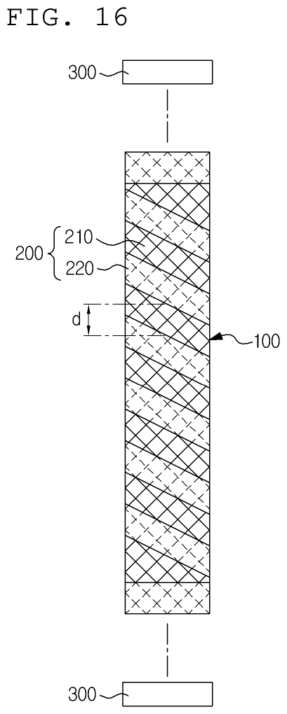

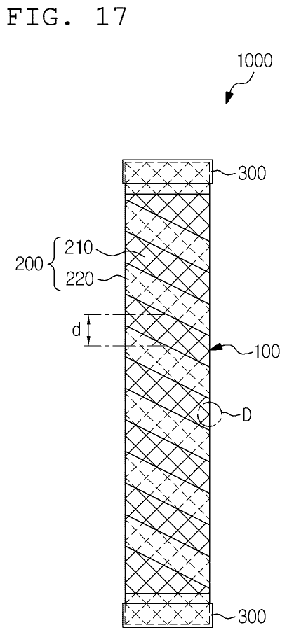

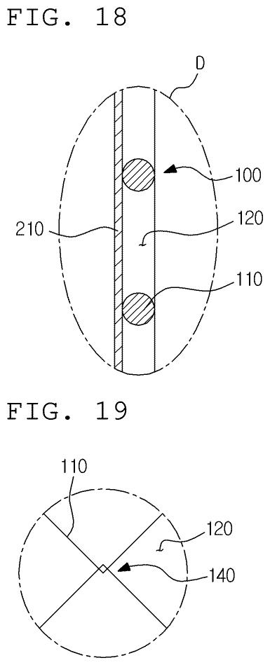

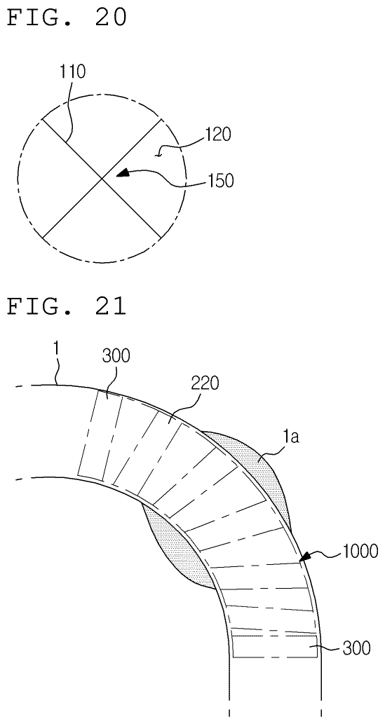

As shown in FIGS. 16 to 22, according to a first modified example of the embodiment of the present disclosure, the second PTFE tape 220 of the covered stent 1000 is formed to have a helical shape formed on the outer surface of the cylindrical stent 100 to form a distance d between one and adjacent another turns of second PTFE tape 220, and to cover both ends of the outer surface of the cylindrical stent 100.

At this point, a space part of the space parts 120 located where the first and second PTFE tapes 210 and 220 intersect is blocked, and a space part of the space parts 120 located where the first and second PTFE tapes 210 and 220 do not intersect is not blocked.

Furthermore, among the hook parts 140 and the cross parts 150, a hook part 140 and a cross part 150 located where the first and second PTFE tapes 210 and 220 do not intersect are free without being fixed to the film part 200.

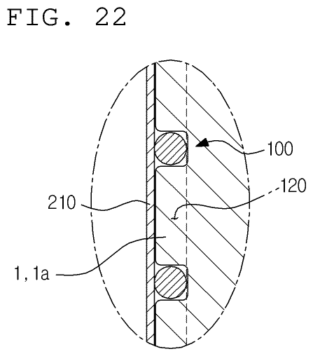

As shown in FIGS. 21 to 22, according to the embodiment of the present disclosure, the covered stent 1000 is inserted into the lumen 1 in the body, such as urethra, pancreatic duct, etc. of the human body, by the stent delivery system, and is used to expand the lesion area la of narrowing or blocking occurring in the lumen 1.

Furthermore, since a hook part 140 and a cross part 150 located where the first and second PTFE tapes 210 and 220 do not intersect are not fixed to the film part 200, the hook part 140 and the cross part 150 are deformed in response to the curved lumen 1 in the body, and the shape of each space part 120 is changed.

For this reason, the covered stent 1000 is deformed into a curved shape in response to the curved lumen 1 in the body.

Furthermore, since some of the space parts 120 located where the first and second PTFE tapes 210 and 220 do not intersect are not blocked, the lumen 1 in the body and the lesion area la are inserted into the space parts 120.

For this reason, the covered stent 1000 is obstructed by the lumen 1 in the body and the lesion area la that are inserted into the space parts 120.

Although the specific embodiments of the present disclosure have been provided, the present disclosure is not limited to the above-described embodiments, and various modifications, equivalents, additions and substitutions are possible by one of ordinary skill in the art to which this present disclosure belongs, without departing from the scope and spirit of the present disclosure.

What is claimed is:

1. A covered stent comprising:
a cylindrical stent (100) formed from shape memory alloy wires (110) that are woven or intersected together into a hollow cylindrical mesh shape to form multiple space parts (120) within the wires (110), and having multiple bent parts (130) formed on each end along circumference;
a film part (200) formed by bonding first and second polytetrafluoroethylene (PTFE) tapes (210 and 220), the tapes being respectively formed on inner and outer surfaces of the cylindrical stent (100), together with heat and pressure; and
a polytetrafluoroethylene (PTFE) protection member (300) provided at each end of the cylindrical stent (100), having a hollow cylindrical shape and having a groove (310) formed at a thickness portion to cover each bent part (130) as an end of the cylindrical stent (100) is inserted into the groove (310), and bonded to an outer portion of the film part (200) with heat and pressure.

2. The covered stent of claim 1, wherein the protection member (300) has a hollow cylindrical shape, and a portion of the protection member (300) is inserted into an inner or outer portion of the cylindrical stent (100) at each end of the cylindrical stent (100), and
a remaining portion of the protection member (300) is folded to form the groove (310) that covers each bent part (130) as an end of the cylindrical stent (100) is inserted, cover each end of the cylindrical stent (100), and bond the protection member (300) to an outer portion of the film part (200) with heat and pressure.

3. The covered stent of claim 1, wherein the second PTFE tape (220) has a helical shape on the outer surface of the cylindrical stent (100) with a distance (d) between turns and is formed to cover each end of the outer surface of the cylindrical stent (100).
